(19) United States
(12) Patent Application Publication
(10) Pub. No.: US 2008/0095857 A1
(43) Pub. Date: Apr. 24, 2008

(54) Carrier system in the form of protein-based nanoparticles for the cell-specific enrichment of pharmaceutically active substances

(75) Inventors: Sabine Balthasar, Kelkheim (DE); Hagen Von Briesen, Hunstetten (DE); Norbert Dinauer, Frankfurt (DE); Jorg Kreuter, Bad Homburg (DE); Klaus Langer, Schoneck (DE); Heidrun Wartlick, Frankfurt (DE)

Correspondence Address: D. PETER HOCHBERG CO. L.P.A., 1940 EAST 6TH STREET, CLEVELAND, OH, 44114, US

(73) Assignee: LTS LOHMANN THERAPIE-SYSTEME AG LOHMANNSTRASSE 2, ANDERNACH (DE)

(21) Appl. No.: 10/590,601

(22) PCT Filed: Mar. 2, 2005
(86) PCT No.: PCT/EP05/02185

§ 371 Date: Aug. 24, 2006

(30) Foreign Application Priority Data
Mar. 9, 2004 (DE) 10 2004 011 776.4

(51) Int. Cl. A61K 9/50; A61K 39/44; A61K 47/48

(52) U.S. Cl. 424/499; 514/773; 424/179.1

(57) ABSTRACT

A carrier system in the form of protein-based nanoparticles for the cell-specific, intracellular enrichment of at least one pharmacologically active substance. The system has structures that are coupled by reactive groups. The structures enable a cell-specific attachment and cellular absorption of the nanoparticles.

### CROSS-REFERENCE TO RELATED APPLICATIONS

This application is a National Stage application of International Application No. PCT/EP2005/002185, filed on Mar. 2, 2005, which claims priority of German application number 10 2004 011 776.4, filed on Mar. 9, 2004.

### BACKGROUND OF THE INVENTION

1. Field of the Invention

The invention relates to a carrier system for pharmaceutically active substances which is suitable for cell-specific enrichment of pharmaceutically active substances and which is present in the form of avidin-modified nanoparticles based on protein, preferably based on gelatine and/or serum albumin, particularly human serum albumin (HSA), to which biotinylated antibodies are bound by formation of a stable avidin-biotin complex and wherein additional bonding of pharmaceutically active substances to the nanoparticles can take place both covalently, or by complex formation via the avidin-biotin system, as well as by incorporation or adsorption.

2. Description of the Prior Art

Nanoparticles are particles of a size between 10 and 1000 nm of artificial or natural macromolecular substances and to which medicinal substances or other biologically active materials can be bound covalently, ionically or adsorptively, or in which said materials can be incorporated.

EP 1 392 255 discloses nanoparticles based on human serum albumin, to which apolipoprotein E is coupled covalently or via an avidin/biotin system to enable the crossing of the blood-brain barrier.

It is, however, a special aim of pharmacotherapy not only to achieve the specific enrichment of a pharmacologically active substance or a therapeutically effective medicinal substance in a specific tissue or organ, as described in EP 1 392 255, but in addition to that even in specific cells.

Unmodified nanoparticles enable passive “drug targeting”, which is characterised by the particles being absorbed by cells of the mononuclear phagocyte system (MPS) following intravascular application. Enrichment of such nanoparticles has been observed in macrophages of the liver, the spleen, the bone marrow, as well as in circulating monocytes. Passive “drug targeting” is distinguished from active “drug targeting”, which aims at the targeted enrichment of the active substance, with the aid of modified nanoparticles, even in primarily inaccessible body compartments or cell systems. To this end, it is necessary to use nanoparticles with hydrophilic surface structures which minimize unspecific interactions with non-target cells, and to equip them with ligands which enable cell-specific enrichment of the nanoparticles. Such ligands are also called “drug targeting ligands”. By using cell-specific nanoparticles as a carrier for medicinal substances it is made possible to enrich a pharmacologically active substance in target cells under controlled conditions, or to transport a pharmacologically active substance specifically to its site of action in the body. Most medicinal substances do not achieve this object without a suitable medicinal form and exhibit, at best, a cellular enrichment or body distribution which is due to the physicochemical properties of the active substance itself. Only part of the active substance applied reaches the desired destination, while the remaining part is responsible for unwanted side effects or toxic effects. Thus, cell-specific nanoparticles contribute to reducing unwanted side effects and toxic properties of active substances.

In initial trials, hydrophilic latex particles were used which had been prepared by copolymerisation of hydroxyethyl methacrylate, methacrylic acid and methyl methacrylate. To these particles was bound an antibody to rabbit γ-globulin. In comparison to unmodified particles, it was observed that the antibody-modified preparation bound to lymphocytes which had been pre-incubated with a rabbit-derived antiserum to these lymphocytes.

Subsequently, corresponding particle systems based on polyacrylates, with ion oxide additionally bound thereto, were used in order to carry out a magnetic separation of lymphocytes and erythrocytes.

On the basis of this basic work, monoclonal anti-CD3 antibodies were then bound via a C7 spacer structure to polyacrylate nanoparticles, and these were examined under cell culture conditions. The problem with these works was, however, that the association of the cells with the subpopulations, and thereby the observed particle association with the corresponding subpopulation, was carried out entirely visually under the microscope and could thus not be made without doubt.

The adsorptive binding of monoclonal antibodies to the surface of polyhexyl cyanoacrylate nanoparticles was examined as well. On the one hand, an effective adsorption of antibodies to the particle surface could be observed, on the other hand the addition of further serum components resulted in a competitive displacement of the antibodies from the particle surface. Insofar, the adsorptive binding of ligands is not suitable for cell-specific drug targeting in biological systems.

A further disadvantage of the described cell-specific nanoparticle systems is the fact that they are based on polymer materials, such as latex and polyacrylates, that are not biologically degradable.

Initial trials on the protein-chemical binding of antibodies to the surface of serum albumin-based nanoparticles have been made. In these trials, the antibodies were conjugated via the primary amino groups of the albumin and of the antibodies, using the glutaraldehyde reaction. As ligands, monoclonal antibodies to Lewis lung carcinoma as well as, by comparison, unspecific IgG antibodies were employed. Although the free specific antibody exhibited a clear enrichment in the target cells both under cell culture conditions and after intravenous application to test animals, after conjugation with the nanoparticles only a very low enrichment of the particles was detected in the tumour under in vivo conditions. The main portion of the applied nanoparticles was found in the liver and the kidneys. Nanoparticles which were conjugated with the unspecific IgG antibody showed no enrichment in the tumour tissue whatsoever. Under the experimental conditions selected, it was thus only possible to achieve a low specificity of the conjugated nanoparticles based on human serum albumin. The main part of this particle system exhibited the unspecific body distribution typical for passive drug targeting. However, since the conjugated nanoparticles employed were only insufficiently characterized with regard to the binding of the antibodies, it remains unclear whether the lack of specificity was caused by insufficient antibody binding. In any case, to date no evidence has been produced for a specific and receptor-mediated absorption of nanoparticles in target cells with simultaneous circumvention of non-target cells.

### SUMMARY OF THE INVENTION

It was thus the object of the present invention to provide nanoparticles that do not have the disadvantages of the above-described nanoparticle systems, but show a high cell specificity even when used in biological systems in order to enable enrichment of pharmacologically active substances specifically in selected target cells, and that are based on a biologically degradable material.

The object is achieved, surprisingly, by a carrier system in the form of avidin-modified protein-based nanoparticles to which biotinylated antibodies are bound by forming a stable avidin-biotin complex. Preferably, gelatine and/or serum albumin, especially preferably human serum albumin, is/are used as proteins. With these modified nanoparticles, additional bonding of pharmacologically active substances to the nanoparticles can take place both covalently, by complex formation via the avidin-biotin system, as well as by incorporation or adsorption.

### BRIEF DESCRIPTION OF THE DRAWINGS

FIG. 1 shows the structure of an avidin-modified nanoparticle based on gelatine or HSA, with an antibody bound by means of the avidin-biotin complex.

FIG. 2 is a bar chart showing the cellular absorption of antibody (Trastazumab)-modified gelatine A nanoparticles in various breast cancer cell lines, determined by FACS analysis. The antibody-modified nanoparticles were in each case compared with the non-modified nanoparticles under the same incubation conditions. Untreated cells served as control.

### DETAILED DESCRIPTION OF THE PRESENT INVENTION

To prepare nanoparticles according to the present invention, an aqueous gelatine solution was converted, by a double desolvation procedure, to nanoparticles, and the latter were subsequently stabilized by crosslinking. The functional groups (amino groups, carboxyl groups, hydroxyl groups) located on the surface of these nanoparticles can be converted to reactive thiol groups by suitable reagents. Functional proteins can be bound to these thiol group-modified nanoparticles by bifunctional spacer molecules which are reactive both to amino groups and to free thiol groups. These functional proteins include, in particular, avidin derivatives or cell-specific antibodies.

When preparing the nanoparticles for the cell culture tests described hereinbelow, the primary amino groups on the particle surface were reacted with 2-iminothiolane, which resulted in the introduction of free thiol groups on the particle surface. The amino groups of the avidin derivative NeutrAvidin® were activated with the bifunctional spacer Sulfo-MBS (m-maleimidobenzoyl-N-hydroxysulfosuccinimide ester), and after column-chromatographic purification of this activation intermediate stage the thiolated gelatine nanoparticles were added thereto. This intermediate product of the avidin-modified nanoparticles represents a universal carrier system for a variety of biotinylated substances which can be bound via the avidin-biotin complex formation.

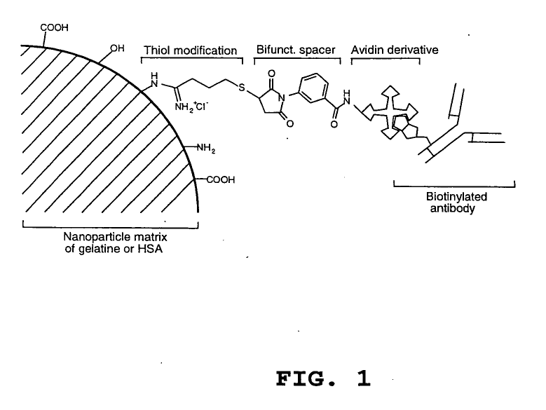

For bonding of the antibodies, preferably of monoclonal antibodies, the antibodies were either purchased in biotinylated form, or they were biotinylated by conversion with NHS biotin (N-hydroxysuccinimidobiotin), and the avidin-modified nanoparticles were added thereto. Thereby, antibody-modified nanoparticles based on gelatine were obtained via the above-described avidin-biotin complex formation (FIG. 1). Corresponding antibody-modified nanoparticles may, however, also be prepared on the basis of serum albumin, preferably human serum albumin.

The present invention thus comprises a carrier system for the cell-specific, intracellular enrichment of at least one pharmacologically active substance, which carrier system is present in the form of protein-based nanoparticles and comprises structures that are coupled by reactive groups, said structures enabling a cell-specific attachment and cellular absorption of the nanoparticles. Gelatine and/or serum albumin, especially preferably human serum albumin, are preferably taken into consideration as the protein basis. The reactive group preferably is an amino, thiol, carboxyl group, or an avidin derivative, and the coupled structure is an antibody, especially preferably a monoclonal antibody.

The invention also encompasses a corresponding carrier system which additionally contains at least one pharmaceutically active substance that is bound by adsorption, incorporation or covalent or complexing bonds to the carrier system or nanoparticles by the reactive groups.

The invention further encompasses the use of a carrier system according to the invention for producing a medicament for enrichment of a pharmaceutically active substance to or into specific cells.

The invention further encompasses a method for producing a carrier system in the form of protein-based nanoparticles for the cell-specific enrichment of at least one pharmaceutically active substance which comprises the following steps:
desolvating an aqueous protein solution;
stabilising the nanoparticles formed by the desolvation by crosslinking;
converting part of the functional groups on the surface of the stabilized nanoparticles to reactive thiol groups;
covalently attaching functional proteins, preferably avidin, by bifunctional spacer molecules;
if required, biotinylating the antibody;
loading the avidin-modified nanoparticles with the biotinylated antibody; and
loading the avidin-modified nanoparticles with a biotinylated and pharmaceutically or biologically active substance.

With the method according to the invention, the use of gelatine and/or serum albumin, especially serum albumin of human origin, is especially preferred.

Preferably, desolvation is carried out by stirring and the addition of a water-miscible non-solvent for proteins, or by salting-out. The water-miscible non-solvent for proteins is preferably selected from the group consisting of ethanol, methanol, isopropanol and acetone.

To stabilise the nanoparticles, thermal processes or bifunctional aldehydes, especially glutaraldehyde or formaldehyde, are used with preference.

As the thiol group-modifying agent, preferably a substance is used which is selected from the group consisting of 2-iminothiolane, a combination of 1-ethyl-3-(3-dimethylaminopropyl)carbodiimide and cysteine, or a combination of 1-ethyl-3-(3-dimethylaminopropyl)carbodiimide and cystaminium dichloride as well as dithiotreitol.

As the bifunctional spacer molecule, preferably a substance is used that is selected from the group consisting of m-maleimidobenzoyl-N-hydroxysulfosuccinimide ester, sulfosuccinimidyl-4-[N-maleimidomethyl]cyclohexane-1-carboxylate, sulfosuccinimidyl-2-[m-azido-o-nitrobenzamido]-ethyl-1,3′dithiopropionate, dimethyl-3,3′-dithiobispropionimidate-dihydrochloride and 3,3′-dithiobis[sulfosuccinimidyl propionate].

### EXAMPLE

To prepare protein nanoparticles, 500 mg gelatine A was dissolved in 10.0 ml purified water while heating, and precipitated to a sediment by adding 10.0 ml acetone. The precipitated gelatine was separated, redissolved in 10.0 ml water while heating, and the pH value of the solution was adjusted to pH 2.5. Nanoparticles were obtained from this solution by dropwise addition of 30 ml acetone (desolvation process).

The nanoparticles were stabilised by adding 625 μl glutaraldehyde 8% and stirring over night. The nanoparticles were purified in aliquots of 2.0 ml by 5 cycles of centrifugation and redispersion by ultrasound treatment. For thiolation of the particle surface, 2.5 ml of a solution of 30 mg 2-iminothiolane (Traut's reagent) in Tris-buffer pH 8.5 was added to 1.0 ml of nanoparticle suspension (20 mg/ml), and this was stirred for 24 hours. Following the thiolation, the purification as described above was repeated.

The avidin derivative FITC-NeutrAvidin® was coupled with the thiolated nanoparticles via the bifunctional spacer Sulfo-MBS (m-maleimidobenzoyl-N-hydroxysulfosuccinimide ester). To activate the avidin derivative, 0.75 mg sulfo-MBS was added to a solution of 2.5 mg FITC-NeutrAvidin® in 500 μl PBS buffer pH 7.0, and this was stirred for 1 hour at room temperature. The separation of unreacted sulfo-MBS from the activated NeutrAvidin® was made by size exclusion chromatography. Those fractions wherein NeutrAvidin® was detected by a spectrophotometric detection at 280 nm were combined, the suspension of the thiolated nanoparticles was added thereto, and this was stirred for 12 hours at room temperature. A further purification of the now covalently FITC-NeutrAvidin®-modified nanoparticles was performed as described above. The supernatants obtained from the particle purification were photometrically examined for unbound NeutrAvidin®, and the portion of covalently bound NeutrAvidin® was calculated therefrom. The functionality of the bound NeutrAvidin®, expressed as the number of the biotin binding sites per avidin molecule, was determined by a titration experiment with biotin-4-fluorescein. It was shown that 2.4 of the 4 biotin binding sites theoretically present in the avidin molecule are also functionally available after the conjugation with the nanoparticles. For loading with the antibodies, 500 μl of the biotinylated antibodies (25 μg/ml) were added to 150 μl of the NeutrAvidin®-modified nanoparticles (20 mg/ml), followed by incubation for 90 minutes at 10° C.

After incubation, the particles were again purified by centrifugation and redispersion. The resultant particle supernatants were examined for unbound antibodies by Western-Blot analysis. It was shown that more than 80% of the antibody employed was present bound to the particle system.

With the aid of the described particle system, cell-specific particle enrichments were found in different cell culture tests in target cells which carried the surface antigen recognised by the antibody. The following cell culture models were used:

Lymphocytic target cells (Jurkat T cells) with the surface antigen CD3.

Nanoparticles were loaded with a biotinylated anti-CD3 antibody.

Human breast cancer cell lines (SK-Br-3-, MCF-7-, BT474 cells) with expression of the HER2 surface antigen)

Nanoparticles were loaded with the approved antibody Trastuzumab (Herceptin®), which had previously been biotinylated.

The cultured cells were incubated with the nanoparticle system in concentrations between 100 and 1000 μg/ml, and after an incubation time of 4 hours unbound nanoparticles were separated by washing the cells. The cells were examined by flow cytometry (FACS) as well as confocal microscopy (CLSM) with regard to nanoparticle absorption.

For the experiments on the cell-specific absorption of the biotinylated-anti-CD3-antibody-modified nanoparticles in lymphocytic cells, Jurkat-T cells were sown in a density of 1×10⁶ cells per well onto a 24-well microtitre plate and cultured in RPMI medium. The medium was supplemented with 10% (vol/vol) fetal calf serum (FCS), 2% L-glutamine and 1% penicillin/streptomycin. The nanoparticles modified with the antibody were incubated with the cells at a concentration of 1000 μg/ml for a period of 4 hours. To prove a specific cellular absorption via the T cell receptor, different control experiments were performed. On the one hand, nanoparticles were used which were loaded with unspecific IgG antibodies instead of the specific anti-CD3 antibodies. Furthermore, the experiments were performed with Jurkat T cells which were preincubated with 2.5 μg free IgG or anti-CD3 antibodies per 1×10⁶ cells for 30 minutes. After this period, the nanoparticles loaded with the anti-CD3 antibody were added. On the other hand, comparative experiments were carried out using MCF-7 cells which did not have the CD3 surface antigen. The cellular absorption was evaluated qualitatively by confocal microscopy as well as quantitatively by flow cytometry.

For the experiments on the cell-specific absorption of the biotinylated-anti-HER2-antibody-modified nanoparticles in breast cancer cells, HER2-overexpressing cells (BT474 and SK-Br-3) were sown in a density of 2×10⁵, respectively 1×10⁵, cells per well onto a 24-well microtitre plate and cultured in RPMI medium and McCoy's 5 A, respectively. The medium of the BT474 was supplemented with 20% (vol/vol) fetal calf serum (FCS), 2% L-glutamine, 1% penicillin/streptomycin and 100 U insulin. The medium of the SK-Br-3 was supplemented with 10% (vol/vol) fetal calf serum (FCS), 2% L-glutamine and 1% penicillin/streptomycin. The antibody-modified nanoparticles were incubated with the cells at a concentration of 100 μg/ml for a period of 3 hours.

To prove a specific cellular absorption via the HER2 receptor, different comparison experiments were performed. On the one hand, nanoparticles were used which were not loaded with a specific antibody. On the other hand, the experiments were made with MCF-7 cells (normal BER2 expression). Furthermore, control experiments were carried out with SK-Br-3 cells which were pre-incubated for 30 minutes with 2.5 μg/ml free anti-BER2 antibodies (Trastuzumab) per 2×10⁵ cells. After this period, the nanoparticles loaded with the anti-HER2 antibody were added. The cellular absorption was evaluated qualitatively by confocal microscopy as well as quantitatively by flow cytometry.

### RESULTS

Lymphocytic Target Cells (Jurkat T Cells)

It was shown both by FACS and CLSM that nanoparticles were cellularly absorbed which were used in a form modified with the cell-specific anti-CD3 antibody. The cellular absorption could be avoided where the cells were treated with the free specific antibody prior to adding the particles. Pretreatment with free unspecific IgG antibodies, however, did not reveal any influence on particle absorption. Modification of the nanoparticles with an unspecific IgG antibody instead of the specific anti-CD3 antibody likewise did not lead to absorption in the target cells. Control experiments were furthermore performed with breast cancer cells (NCF-7 cells) which did not have the CD3 surface antigen. In these control experiments no absorption of the nanoparticle preparations was observed under any of the selected conditions.

Human Breast Cancer Cell Lines (SK-Br-3-, MCF-7-. BT474 Cells)

The cells which were used showed to a different extent an expression of the HER2 surface antigen, which was used as point of attack for cellular absorption of the antibody-modified nanoparticles. Expression of the cells was determined prior to incubation with the nanoparticles by Western-Blot analysis (Table 1).

TABLE 1
Expression of the HER2 surface antigen on the surface of
different tumour cells determined by Western-Blot analysis.
Expression was calculated relative to the values of
“normally expressing” MCF-7 cells.

| | Expression HER2 |
|---|---|
| Cell line | [%] |
| BT474 | 311 |
| MCF-7 | 100 |
| SK-Br-3 | 366 |

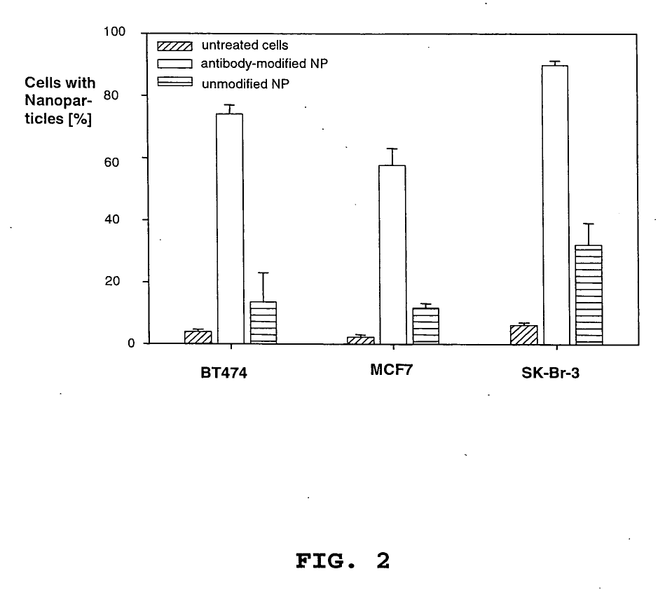

Both by FACS as well as CLSM, it could be shown that nanoparticles were cellularly absorbed which were used in the form modified with the cell-specific antibody Trastuzumab (FIG. 2). The cellular absorption of the specific nanoparticles could be prevented where the cells were treated with the free specific antibody prior to addition of the particles. Nanoparticles of the same batch which were not used in the form modified with the biotinylated antibody exhibited only a low cellular enrichment under the conditions selected. The extent of the cellular absorption of the antibody-modified nanoparticles could be correlated with the extent of the expression of the HER2 surface antigen.

The results of the aforementioned cell culture experiments clearly show that antibody-modified nanoparticles based on gelatine enable a specific enrichment in the target cells. Under comparable conditions the particle systems are absorbed only in the corresponding target cells, but not in control cells. The preincubations with free specific antibody clearly show that particle absorption takes place via a process of receptor-mediated endocytosis. Thus, the nanoparticulate medicinal agent carrier system which has been developed affords the possibility of transporting medicinal substances specifically to diseased cells, provided that these target cells differ in their surface properties from healthy cells.

With the antibody-modified nanoparticles based on gelatine according to the invention there is provided a well-characterized, particulate carrier system which, by a functional drug targeting ligand carried on the surface of said carrier system, enables a cell-specific absorption and enrichment even of such pharmaceutically active substances as are bound to the carrier system by adsorption, incorporation or by covalent or complex-forming bonds.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A carrier system for the cell-specific, intracellular enrichment of at least one pharmacologically active substance, wherein said carrier system is present in the form of protein-based nanoparticles and comprises structures and reactive groups, wherein said reactive groups couple said structures to form a coupled structure, said structures enabling a cell-specific attachment and cellular absorption of the nanoparticles.

2. The carrier system according to claim 1, wherein the reactive group is selected from the group consisting of an amino group, a thiol group, a carboxyl group and an avidin derivative.

3. The carrier system according to claim 1, wherein the coupled structure is an antibody.

4. The carrier system according to claim 3, wherein the antibody is a monoclonal antibody.

5. The carrier system according to claim 1, wherein said carrier system additionally comprises a pharmaceutically active substance that is bound to the carrier system by the reactive groups by a method selected from the group consisting of adsorption, incorporation, covalent bonds and complexing bonds.

6. Use of a carrier system according to claim 1 for producing a medicament for enrichment of a pharmaceutically active substance to/in specific cells.

7. A method for producing a carrier system in the form of protein-based nanoparticles for the cell-specific enrichment of at least one pharmacologically active substance, wherein said method comprises the following steps:
desolvating an aqueous protein solution to form nanoparticles;
stabilising the nanoparticles formed by the desolvation step by crosslinking;
converting part of the functional groups on the surface of the stabilised nanoparticles to reactive thiol groups;
covalently attaching functional proteins by bifunctional spacer molecules;
if required, biotinylating the antibody;
loading the functional-protein-modified nanoparticles with the biotinylated antibody; and
loading the functional-protein-modified nanoparticles with a biotinylated and pharmaceutically or biologically active substance.

8. The method according to claim 7, wherein the protein base is selected from the group consisting of gelatine and serum albumin.

9. The method according to claim 7, wherein the desolvation step is carried out by a method selected from the group consisting of stirring and adding a water-miscible non-solvent for proteins and by salting-out.

10. The method according to claim 9, wherein the water-miscible non-solvent for proteins is selected from the group consisting of ethanol, methanol, isopropanol and acetone.

11. The method according to claim 7, wherein said step of stabilising the nanoparticles is achieved by utilising at least one method selected from the group consisting of thermal processes, bifunctional aldehydes and formaldehyde.

12. The method according to claim 11, wherein said bifunctional aldehyde is glutaraldehyde.

13. The method according to claim 7, further comprising the step of using a substance as the thiol group-modifying agent that is selected from the group consisting of 2-iminothiolane, a combination of 1-ethyl-3-(3-dimethylaminopropyl)carbodiimide and cysteine, a combination of 1-ethyl-3-(3-dimethylaminopropyl)-carbodiimide and cystaminium dichloride and as well as dithiotreitol.

14. The method according to claim 7, further comprising the step of using a substance as a bifunctional spacer molecule that is selected from the group consisting of m-maleimidobenzoyl-N-hydroxysulfosuccinimide ester, sulfosuccinimidyl-4-[N-maleimido-methyl]cyclohexan-1-carboxylate, sulfosuccinimidyl-2-[m-azido-o-nitrobenzamido]-ethyl-1,3′dithiopropionate, dimethyl-3,3′-dithiobispropionimidate-dihydrochloride and 3,3′-dithiobis[sulfosuccinimidylpropionate].

15. The carrier system according to claim 1, wherein said protein-based nanoparticles are based on at least one protein selected from the group consisting of gelatine and serum.

16. The carrier system according to claim 16, wherein said protein-based nanoparticles are based on human serum albumin.

17. The method according to claim 7, wherein said functional proteins are avidin.

18. The method according to claim 8, wherein the protein base is human serum albumin.
